(19) United States
(12) Patent Application Publication
(10) Pub. No.: US 2016/0106881 A1
(43) Pub. Date: Apr. 21, 2016

(54) PROCESS TO MAKE WATER-ABSORBING POLYMER STRUCTURE HAVING SUPERABSORBENT POLYMER INDEX

(71) Applicant: EVONIK DEGUSSA GMBH, Essen (DE)

(72) Inventors: Jörg Harren, Krefeld (DE); Stephan Ramlow, Krefeld (DE); Axel Busch, Krefeld (DE); Franck Furno, Clementi (SG)

(73) Assignee: Evonik Degussa GmbH

(21) Appl. No.: 14/976,659

(22) Filed: Dec. 21, 2015

Related U.S. Application Data
(63) Continuation of application No. 12/525,955, filed on Sep. 4, 2009.

(30) Foreign Application Priority Data
Feb. 9, 2007 (DE) 102007007203.3

(51) Int. Cl. A61L 15/46; A61L 15/24; A61L 15/60; A61L 15/18; A61L 15/20

(52) U.S. Cl. CPC A61L 15/46; A61L 15/18; A61L 15/20; A61L 15/60; A61L 15/24

(57) ABSTRACT

The present invention relates to a process for the production of a water-absorbing polymer structure, comprising the process steps:
i) providing an untreated water-absorbing polymer structure having a degree of neutralization of at most 70 mol %;
ii) bringing the untreated water-absorbing polymer structure into contact with an acidic component.

The invention also relates to the water-absorbing polymer structures obtainable by this process, water-absorbing polymer structures, a composite, a process for the production of a composite, the composite obtainable by this process, foams, shaped articles, fibers, foils, films, cables, sealing materials, liquid-absorbing hygiene articles, carriers for plant and fungal growth-regulating agents, packaging materials, soil additives or building materials, and the use of a water-absorbing polymer structure.

The present invention relates to a process for the preparation of a water-absorbing polymer structure, the water-absorbing polymer structures obtainable by this process, water-absorbing polymer structures, a composite, a process for the production of a composite, the composite obtainable by this process, foams, shaped articles, fibers, foils, films, cables, sealing materials, liquid-absorbing hygiene articles, carriers for plant and fungal growth-regulating agents, packaging materials, soil additives or building materials, and the use of a water-absorbing polymer structure.

Superabsorbers are water-insoluble crosslinked polymers which are capable of taking up, with swelling and formation of hydrogels, large amounts of water and aqueous liquids, in particular body fluids, preferably urine or blood, and of retaining them under pressure. Superabsorbers absorb preferably at least 100 times their own weight of water. Further details of superabsorbers are disclosed in “Modern Superabsorbent Polymer Technology”, F. L. Buchholz, A. T. Graham, Wiley-VCH, 1998. Due to these characteristic properties, these water-absorbing polymers are chiefly incorporated into sanitary articles, such as, for example, baby nappies, incontinence products or sanitary towels.

Superabsorbers are as a rule prepared by free-radical polymerization of monomers which carry acid groups in the presence of crosslinking agents, these monomers which carry acid groups being at least partly neutralized before or also after the polymerization.

In the case of hygiene articles, especially in the field of adult hygiene, there is a need for superabsorbers with the ability to bind unpleasant odors. Such odor-binding properties are rendered possible, for example, by addition of odor-binding additives, such as, for example, cyclodextrins, as is described, for example, in WO 01/13841 A1. The disadvantage of addition of odor-binding additives is, however, that these as a rile pulverulent additives often lead to dusting of the water-absorbing polymer structures, especially during conveying in installations for the production of hygiene articles. Further possibilities for odor control in hygiene articles are described, for example, in WO 03/002623 A1.

Binding of the ammonia formed by bacterial breakdown of urea is important in particular for the wearing comfort of hygiene articles. Due to their chemical composition from crosslinked, partly neutralized acrylic acid, superabsorbers are capable of binding ammonia by acid-base reactions. By the lowering of the degree of neutralization of the polymerized acrylic acid and the associated increase in protonated carboxylic acid groups, the ammonia-binding capacity can be increased. However, limits are imposed on the lowering of the pH, since as the degree of neutralization decreases, the overall performance of a superabsorbent polymer structure deteriorates. Modern superabsorbers have an optimum liquid absorption performance at degrees of neutralization in the range of from 65 to 80 mol %.

Down to a degree of neutralization of 50 mol %, an acceptable overall performance can still be achieved. A further disadvantage of a degree of neutralization of 50 mol % or less is that the swelling properties are greatly reduced due to the low ionicity of the polymer network.

To improve further the odor-binding properties in hygiene articles by the lowering of the pH, U.S. Pat. No. 3,794,034 proposes providing a pulverulent acidic substance, such as, for example, citric acid, within the fiber material of the hygiene article. The use of, for example, superabsorbers based on crosslinked polyacrylates is not disclosed in U.S. Pat. No. 3,794,034.

WO 00/35502 A1 proposes, for improving the odor-binding properties of a hygiene article comprising a superabsorber, also adding to the absorbent core of such a hygiene article, in addition to the superabsorber, bacteria which produce lactic acid so that after the hygiene article comes into contact with body fluids, the pH is in a range of from 3.5 to 5.5. The disadvantage of such a hygiene article is that on the one hand the addition of bacteria which produce lactic acid necessitates an additional process step in the production of the hygiene articles, and that on the other hand, in particular due to the temperature sensitivity of the bacteria which produce lactic acid, the odor-binding properties of such hygiene articles can be adversely influenced by environmental influences, in particular by particularly high or low temperatures.

WO 01/32226 A1 proposes, for improving the odor-binding properties of a hygiene article comprising a superabsorber, provision of acidic substances, such as, for example, organic carboxylic acids, separated from the superabsorber in the absorbent core of the hygiene article. Here also there is the disadvantage that additional process steps in which the acidic components must be introduced into the absorbent core are necessary in the production of the hygiene article. The odor-binding properties of such hygiene articles moreover are still in need of improvement.

WO 03/002623 A1 describes a process for the preparation of odor-binding superabsorbers in which weakly partly neutralized water-absorbing polymer structures having a pH of less than 5.7 are post-crosslinked on the surface. The disadvantage of the superabsorbers described in this prior art is, however, that the ammonia-binding capacity is only low.

The present invention was based on the object of improving the disadvantages resulting from the prior art with respect to the odor-binding properties of hygiene articles comprising superabsorbers.

In particular, the present invention was based on the object of providing water-absorbing polymer structures which, compared with the water-absorbing polymer structures known from the prior art, are better capable of suppressing the escape of unpleasantly smelling compounds from hygiene articles and nevertheless have satisfactory absorption properties.

The present invention was moreover based on the object of providing water-absorbing polymer structures which have improved odor-binding properties compared with conventional polymer structures and in addition can be processed better compared with these conventional superabsorbers, in particular can be transported better in conveying installations for the production of hygiene articles.

The present invention was also based on the object of providing a process with which such advantageous water-absorbing polymer structures can be prepared.

The present invention was also based on the object of providing composites which, compared with the composites known from the prior art, have improved odor-binding properties and absorption properties and in addition can be prepared with as few process steps as possible compared with conventional composites.

A contribution towards achieving the above-mentioned objects is made by a process for the preparation of a water-absorbing polymer structure comprising the process steps:
i) providing an untreated, water-absorbing polymer structure having a degree of neutralization of at most 70 mol %, preferably of at most 65 mol %, still more preferably of at most 60 mol %, more preferably of at most 55 mol %, most preferably having a degree of neutralization in a range of from 45 to 55 mol %, the degree of neutralization preferably not falling below 20 mol %, particularly preferably 30 mol %, still more preferably 40 mol % and most preferably 45 mol %;
ii) bringing the water-absorbing polymer structure into contact with an acidic, preferably organic component.

It has been found, surprisingly, that by mixing of an only weakly neutralized water-absorbing polymer structure with an acidic component, water-absorbing polymer structures which at the same time have advantageous odor-binding properties and an advantageous overall performance can be obtained.

“Untreated” in the context of the present invention means that the water-absorbing polymer structures provided in process step i) have not yet been brought into contact with the acidic, preferably organic component. On the other hand, the term “untreated” does not rule out that the water-absorbing polymer structures can be modified by means of other surface modification measures, such as, for example, surface post-crosslinking.

Preferred untreated water-absorbing polymer structures provided in process step i) are fibers, foams or particles, fibers and particles being preferred and particles being particularly preferred.

Polymer fibers which are preferred according to the invention have dimensions such that they can be incorporated into or as yarns for textiles and also directly into textiles. It is preferable according to the invention for the polymer fibers to have a length in the range of from 1 to 500 mm, preferably 2 to 500 mm and particularly preferably 5 to 100 mm and a diameter in the range of from 1 to 200 denier, preferably 3 to 100 denier and particularly preferably 5 to 60 denier.

Polymer particles which are preferred according to the invention have dimensions such that they have an average particle size in accordance with ERT 420.2-02 in the range of from 10 to 3,000 gm, preferably 20 to 2,000 gm and particularly preferably 150 to 850 gm or 150 to 600 gm. In this context, it is particularly preferable for the proportion of polymer particles having a particle size in a range of from 300 to 600 gm to be at least 30 wt. %, particularly preferably at least 40 wt. % and most preferably at least 50 wt. %, based on the total weight of the post-crosslinked water-absorbing polymer particles.

In a preferred embodiment of the water-absorbing polymer structures provided in process step i), these are based on
(a1) 20-99.999 wt. %, preferably 55-98.99 wt. % and particularly preferably 70-98.79 wt. % of polymerized, ethylenically unsaturated monomers carrying acid groups, or salts thereof, or polymerized, ethylenically unsaturated monomers containing a protonated or quaternized nitrogen, or mixtures thereof, mixtures comprising at least ethylenically unsaturated monomers containing acid groups, preferably acrylic acid, being particularly preferred,
(a2) 0-80 wt. %, preferably 0-44.99 wt. % and particularly preferably 0.1-44.89 wt. % of polymerized, monoethylenically unsaturated monomers which can be copolymerized with (a1),
(a3) 0.001-5 wt. %, preferably 0.01-3 wt. % and particularly preferably 0.01-2.5 wt. % of one or more crosslinking agents,
(a4) 0-30 wt. %, preferably 0-5 wt. % and particularly preferably 0.1-5 wt. % of a water-soluble polymer,
(a5) 0-20 wt. %, preferably 2.5-15 wt. % and particularly preferably 5-10 wt. % of water, and
(a6) 0-20 wt. %, preferably 0-10 wt. % and particularly preferably 0.1-8 wt. % of one or more auxiliary substances, the sum of the amounts by weight (a1) to (a6) being 100 wt. %.

In this connection, the requirement according to which the untreated water-absorbing polymer structures provided in process step i) are to have a degree of neutralization of at most 70 mol %, preferably of at most 65 mol %, still more preferably of at most 60 mol %, more preferably of at most 55 mol % and most preferably a degree of neutralization in a range of from 45 to 55 mol % means that at most 70 mol %, at most 65 mol %, at most 60 mol % or, respectively, at most 55 mol % of the acid groups of the monomers (a1) are present as deprotonated carboxylate groups.

The neutralization can also be carried out in part or entirely after the polymerization. The neutralization can furthermore be carried out with alkali metal hydroxides, alkaline earth metal hydroxides, ammonia and carbonates and bicarbonates. In addition, any further base which forms a water-soluble salt with the acid is conceivable. Mixed neutralization with various bases is also conceivable. Neutralization with ammonia and alkali metal hydroxides is preferred, particularly preferably with sodium hydroxide and with ammonia.

Preferred ethylenically unsaturated monomers (a1) containing acid groups are preferably those compounds which are mentioned as ethylenically unsaturated monomers (a1) containing acid groups in WO 2004/037903 A2, which is introduced herewith as reference and is thus part of the disclosure. Particularly preferred ethylenically unsaturated monomers (a1) containing acid groups are acrylic acid and methacrylic acid, acrylic acid being most preferred.

According to one embodiment of the process according to the invention, untreated water-absorbing polymer structures in which the monoethylenically unsaturated monomers (a2) which can be copolymerized with (a1) are acrylamides, methacrylamides or vinylamides are employed.

Preferred (meth)acrylamides are, in addition to acrylamide and methacrylamide, alkyl-substituted (meth)acrylamides or aminoalkyl-substituted derivatives of (meth)acrylamide, such as N-methylol(meth)acrylamide, N,N-dimethylamino(meth)acrylamide, dimethyl(meth)acrylamide or diethyl(meth)acrylamide. Possible vinylamides are, for example, N-vinylamides, N-vinylformamides, N-vinylacetamides, N-vinyl-N methylacetamides, N-vinyl-N-methylformamides and vinylpyrrolidone. Among these monomers, acrylamide is particularly preferred.

According to another embodiment of the process according to the invention, water-absorbing polymer structures in which the monoethylenically unsaturated monomers (a2) which can be copolymerized with (a1) are water-soluble monomers are employed. In this connection, alkoxypolyalkylene oxide (meth)acrylates, such as methoxypolyethylene glycol(meth)acrylates, are preferred in particular.

Water dispersible monomers are furthermore preferred as monoethylenically unsaturated monomers (a2) which can be copolymerized with (a1). Preferred water-dispersible monomers are acrylic acid esters and methacrylic acid esters, such as methyl (meth)acrylate, ethyl (meth)acrylate, propyl (meth)acrylate or butyl (meth)acrylate.

The monoethylenically unsaturated monomers (a2) which can be copolymerized with (a1) furthermore include methylpolyethylene glycol allyl ether, vinyl acetate, styrene and isobutylene.

Crosslinking agents (a3) which are preferably employed are those compounds which are mentioned as crosslinking agents (a3) in WO 2004/037903 A2. Among these crosslinking agents, water-soluble crosslinking agents are particularly preferred. In this context, N,N′-methylenebisacrylamide, polyethylene glycol di(meth)acrylates, triallylmethylammonium chloride, tetraallylammonium chloride and allylnonaethylene glycol acrylate prepared with 9 mol of ethylene oxide per mol of acrylic acid are most preferred.

The polymer structures can comprise as water-soluble polymers (a4) water-soluble polymers such as partly or completely saponified polyvinyl alcohol, polyvinylpyrrolidone, starch or starch derivatives, polyglycols or polyacrylic acid, preferably in a polymerized-in form. The molecular weight of these polymers is not critical, as long as they are water-soluble. Preferred water-soluble polymers are starch or starch derivatives or polyvinyl alcohol. The water-soluble polymers, preferably synthetic polymers, such as polyvinyl alcohol, can also serve as a graft base for the monomers to be polymerized.

Auxiliaries (a6) which are contained in the polymer structures are, preferably, standardizing agents, odor-binding agents, surface-active agents or antioxidants and those additives which have been employed for the preparation of the polymer structures (initiators etc.).

In a particular embodiment of the water-absorbing polymer structures provided in process step i), these are based to the extent of at least 50 wt. %, preferably to the extent of at least 70 wt. % and moreover preferably to the extent of at least 90 wt. % on monomers which carry carboxylic acid groups or carboxylate groups.

The untreated water-absorbing polymer structures can be prepared from the above-mentioned monomers, comonomers, crosslinking agents, water-soluble polymers and auxiliary substances by various polymerization methods. There may be mentioned by way of example in this connection bulk polymerization, which is preferably carried out in kneading reactors, such as extruders, solution polymerization spray polymerization, inverse emulsion polymerization and inverse suspension polymerization.

Solution polymerization is preferably carried out in water as the solvent. The solution polymerization can be carried out continuously or discontinuously. A broad spectrum of possibilities of variation with respect to the reaction circumstances, such as temperatures, nature and amount of the initiators and also of the reaction solution, is to be found from the prior art. Typical processes are described in the following patent specifications: U.S. Pat. No. 4,286,082, DE 27 06 135 A1, U.S. Pat. No. 4,076,663, DE 35 03 458 A1, DE 40 20 780 C1, DE 42 44 548 A1, DE 43 33 056 A1, DE 44 18 818 A1. The disclosures are introduced herewith as reference and therefore form part of the disclosure.

The polymerization is initiated by an initiator as is generally conventional. Initiators which can be used for initiation of the polymerization are all the initiators which form free radicals under the polymerization conditions and are conventionally employed in the preparation of superabsorbers. Initiation of the polymerization by the action of electron beams on the polymerizable aqueous mixture is also possible. Nevertheless, the polymerization can also be initiated in the absence of initiators of the above-mentioned type by the action of high-energy radiation in the presence of photoinitiators. Polymerization initiators can be contained in a solution of monomers according to the invention in dissolved or dispersed form. Possible initiators are all the compounds known to the person skilled in the art which dissociate into free radicals. These include, in particular, those initiators which have already been mentioned as possible initiators in WO 2004/037903 A2.

A redox system comprising hydrogen peroxide, sodium peroxodisulphate and ascorbic acid is particularly preferably employed for preparation of the water-absorbing polymer structures.

Inverse suspension and emulsion polymerization can also be used for preparation of the polymer structures. According to these processes, an aqueous, partly neutralized solution of monomers (a1) and (a2), optionally containing water-soluble polymers and auxiliary substances, is dispersed in a hydrophobic organic solvent with the aid of protective colloids and/or emulsifiers and the polymerization is started by free radical initiators. The crosslinking agents either are dissolved in the monomer solution and are metered together with this, or are added separately and optionally during the polymerization. The addition of a water-soluble polymer (a4) as a graft base is optionally carried out via the monomer solution or by direct initial introduction into the oily phase. The water is then removed azeotropically from the mixture and the polymer is filtered off.

Both in the case of solution polymerization and in the case of inverse suspension and emulsion polymerization, the crosslinking can furthermore be carried out by polymerizing in the polyfunctional crosslinking agent dissolved in the monomer solution and/or by reaction of suitable crosslinking agents with functional groups of the polymer during the polymerization steps. The processes are described, for example, in the publications U.S. Pat. No. 4,340,706, DE 37 13 601 A1, DE 28 40 010 A1 and WO 96/05234 A1, the corresponding disclosure of which is introduced herewith as reference.

The hydrogels obtained after the polymerization in solution polymerization or inverse suspension and emulsion polymerization are dried in a further process step.

In the case of solution polymerization in particular, however, it is preferable for the hydrogels first to be comminuted before the drying. This comminution is carried out by comminution devices known to the person skilled in the art, such as, for example, a meat chopper.

Drying of the hydrogel is preferably carried out in suitable dryers or ovens. Rotary tube ovens, fluidized bed dryers, plate dryers, paddle dryers or infrared dryers may be mentioned by way of example. It is furthermore preferable according to the invention for the drying of the hydrogel to be carried out down to a water content of from 0.5 to 25 wt. %, preferably from 1 to 10 wt. %, the drying temperatures conventionally being in a range of from 100 to 200° C.

The water-absorbing polymer structures obtained after the drying can be ground again in a further process step, especially if they have been obtained by solution polymerization, and sieved to the above-mentioned desired particle size. Grinding of the dried water-absorbing polymer structures is preferably carried out in suitable mechanical comminution devices, such as, for example, a ball mill.

According to a particularly preferred embodiment of the process according to the invention, the untreated water-absorbing polymer structure provided in process step i) is post-crosslink ECL on the surface. Water-absorbing polymer structures post-crosslinked on the surface have a core-shell structure, the polymer structures having a higher degree of crosslinking in the region of the shell than in the core region.

During the surface post-crosslinking, the dried polymer structures or the not yet dried but preferably already comminuted hydrogel is brought into contact with a preferably organic chemical surface post-crosslinking agent. In this context, the post-crosslinking agent, especially if it is not liquid under the post-crosslinking conditions, is preferably brought into contact with the polymer particles or the hydrogel in the form of a fluid Ft comprising the post-crosslinking agent and a solvent. Suitable solvents are, in addition to water, in particular water-miscible organic solvents, such as, for example, methanol, ethanol, 1-propanol, 2-propanol, 1,2-propanediol, 1,3-propanediol, 1-butanol, 2-butanol, tert-butanol or iso-butanol, or mixtures of organic solvents or mixtures of water with one or more of these organic solvents, water being most preferred as the solvent. It is furthermore preferable for the post-crosslinking agent to be contained in the fluid Fi in an amount in a range of from 5 to 75 wt. %, particularly preferably 10 to 50 wt. % and most preferably 15 to 40 wt. %, based on the total weight of the fluid F1.

In the process according to the invention, the polymer structure or the comminuted hydrogel is preferably brought into contact with the fluid F1 comprising the post-crosslinking agent by thorough mixing of the fluid F1 with the polymer structure, suitable mixing units for application of the fluid F1 in turn being the Patterson-Kelley mixer, DRAIS turbulence mixer, Lodige mixer, Ruberg mixer, screw mixers, plate mixers and fluidized bed mixers as well as continuously operating vertical mixers, in which the polymer structure is mixed by means of rotating blades in rapid frequency (Schugi mixer).

During the post-crosslinking, the water-absorbing polymer structure is preferably brought into contact with at most 20 wt. %, particularly preferably with at most 15 wt. %, more preferably with at most 10 wt. %, even still more preferably with at most 5 wt. % of solvent, preferably water, in each case based on the weight of the water-absorbing polymer structure.

In the case of polymer structures in the form of preferably spherical particles, it is furthermore preferable according to the invention for the components to be brought into contact in a manner such that merely the outer region, but not the inner region of the particulate polymer structures is brought into contact with the fluid F, and therefore the post-crosslinking agent.

Compounds which have at least two functional groups which can react with functional groups of a polymer structure in a condensation reaction (=condensation crosslinking agents), in an addition reaction or in a ring-opening reaction are preferably understood as post-crosslinking agents which are employed in the process according to the invention. Those post-crosslinking agents which have been mentioned as crosslinking agents of crosslinking agent class II in WO 2004/037903 A2 are preferred as post-crosslinking agents in the process according to the invention.

Among these compounds, particularly preferred post-crosslinking agents are condensation crosslinking agents, such as, for example, epoxides, such as, for example, ethylene glycol diglycidyl ether or diethylene glycol diglycidyl ether, ethylene glycols, such as, for example, diethylene glycol, triethylene glycol or polyethylene glycol, glycerol, polyglycerol, propylene glycols, such as, for example, dipropylene glycol, tripropylene glycol or polypropylene glycol, diethanolamine, triethanolamine, polyoxypropylene, oxyethylene-oxypropylene block copolymers, sorbitan fatty acid esters, polyoxyethylene sorbitan fatty acid esters, trimethylolpropane, pentaerythritol, polyvinyl alcohol, sorbitol, 1,3-dioxolan-2-one (ethylene carbonate), 4-methyl-1,3-dioxolan-2-one (propylene carbonate), 4,5-dimethyl-1,3-dioxolan-2-one, 4,4-dimethyl-1,3-dioxolan-2-one, 4-ethyl-1,3-dioxolan-2-one, 4-hydroxymethyl-1,3-dioxolan-2-one, 1,3-dioxan-2-one, 4-methyl-1,3-dioxan-2-one, 4,6-dimethyl-1,3-dioxan-2-one and 1,3-dioxolan-2-one.

After the polymer structures or the hydrogels have been brought into contact with the post-crosslinking agent or with the fluid F1 comprising the post-crosslinking agent, they are heated to a temperature in the range of from 50 to 300° C., preferably 75 to 275° C. and particularly preferably 150 to 250° C., so that, preferably as a result of this, the outer region of the polymer structures is more highly crosslinked compared with the inner region (=post-crosslinking). The duration of the heat treatment is limited by the risk that the desired profile of properties of the polymer structures is destroyed as a result of the action of heat.

The surface post-crosslinking described above not only, as just described, can be carried out before process step ii), it is in principle also conceivable to carry out the surface post-crosslinking during or only after process step ii).

In a preferred embodiment, the untreated water-absorbing polymer structure provided in process step i) of the process according to the invention has at least one of the following properties (ERT=EDANA recommended test):
(A) the maximum absorption according to ERT 440.2-02 (in the case of particles, determined for the total particle size fraction) of 0.9 wt. % strength NaCl solution is in a range of from at least 10 to 1,000 g/g, preferably in the range of from 20 to 500 g/g and more preferably in the range of from 50 to 250 g/g,
(B) the extractable content after 16 hours according to ERT 470.2-02 (in the case of particles, determined for the total particle size fraction) is less than 30 wt. %, preferably less than 20 wt. % and more preferably less than 15 wt. %, in each case based on the untreated water-absorbing polymer structure,
(C) the bulk density according to ERT 460.2-02 (in the case of particles, determined for the total particle size fraction) is in the range of from 300 to 1,000 g/l, preferably in the range of from 400 to 900 g/l and more preferably 500 to 800 g/l,
(D) the pH according to ERT 400.2-02 (in the case of particles, determined for the total particle size fraction) of 1 g of the untreated water-absorbing polymer structure in 1 1 of water is less than 6.5, preferably less than 6.0, particularly preferably less than 5.5 and most preferably less than 5.2, the pH preferably not falling below 1.0, particularly preferably 2.0, more preferably 3.0 and most preferably 4.5,
(E) the absorption, determined in accordance with ERT 442.2-02 (in the case of particles, for the total particle fraction), against a pressure of 50 g/cm2 is in a range of from 10 to 26 g/g, preferably in a range of from 13 to 25 g/g and most preferably in a range of from 15 to 24 g/g,
(F) the retention, determined in accordance with ERT 441.2-02 (in the case of particles, for the total particle fraction) and called CRC, is in a range of from 20 to 50 g/g, preferably in a range of from 25 to 40 g/g and most preferably in a range of from 27 to 35 g/g.

According to a particular embodiment of the process according to the invention, polymer structures which are characterized by the following properties or combinations of properties are provided in process step i): (A), (B), (C), (D), (E), (F), (A)(B), (A)(C), (A)(D), (A)(E), (A)(F), (B)(C), (B)(D), (B)(E), (B)(F), (C)(D), (C)(E), (C)(F), (D)(E), (D)(F), (E)(F) and (A)(B)(C)(D)(E)(F), (D) being most preferred.

In process step ii) of the process according to the invention, the untreated water-absorbing polymer structures provided in process step i) are brought into contact with the acidic component, this acidic component preferably being an organic acid. The term “acidic component” in principle also includes compounds which are capable of forming acidic compounds only in the presence of water, such as, for example, acid anhydrides.

Preferred organic acids are monocarboxylic acids, dicarboxylic acids, tricarboxylic acids, carboxylic acid hydrides or mixtures of at least two of these acids.

Among the above-mentioned organic acids, those which are particularly preferred are, in particular, acetic anhydride, maleic anhydride, fumaric anhydride, benzoic acid, formic acid, valeric acid, citric acid, glyoxylic acid, glycollic acid, glycerol phosphoric acid, glutaric acid, chloroacetic acid, chloropropionic acid, cinnamic acid, succinic acid, acetic acid, tartaric acid, lactic acid, pyruvic acid, fumaric acid, propionic acid, 3-hydroxy-propionic acid, malonic acid, butyric acid, isobutyric acid, imidinoacetic acid, malic acid, isothionic acid, methylmaleic acid, adipic acid, itaconic acid, crotonic acid, oxalic acid, salicylic acid, gluconic acid, gallic acid, sorbic acid, gluconic acid and p-oxybenzoic acid, citric acid and tartaric acid being more preferred and citric acid being most preferred. Although in principle the use of organic acids as the acidic component is preferred, the use of inorganic acids or acid anhydrides, such as, for example, P205, SO2, N20, H2SO4 or HCl, as the acidic component in process step ii) is nevertheless conceivable.

The acidic component is preferably brought into contact with the untreated water-absorbing polymer structure in process step ii) of the process according to the invention by mixing the two components, suitable mixing units for this being, in particular, the Patterson-Kelley mixer, DRAIS turbulence mixer, Lodige mixer, Ruberg mixer, screw mixers, plate mixers and fluidized bed mixers or continuously operating vertical mixers, in which the polymer structure is mixed by means of rotating blades in rapid frequency (Schugi mixer).

The acidic component can furthermore be brought into contact with the untreated water-absorbing polymer structure in the form of a fluid F2 comprising a solvent and the acidic component dissolved or dispersed in this solvent, or in the dry form as a powder, the acidic component particularly preferably being brought into contact in the form of a fluid F2. Suitable solvents are in turn, in addition to water, in particular water-miscible organic solvents, such as, for example, methanol, ethanol, 1-propanol, 2-propanol, 1,2-propanediol, 1,3-propanediol, 1-butanol, 2-butanol, tert-butanol, isobutanol or mixtures of organic solvents or mixtures of water with one or more of these organic solvents, water being most preferred as the solvent. If the untreated water-absorbing polymer structure is brought into contact with the fluid F2 comprising the solvent and the acidic component, it is furthermore preferable for this fluid F2 to comprise the acidic component in an amount in a range of from 0.1 to 75 wt. %, particularly preferably 20 to 65 wt. % and most preferably 30 to 60 wt. %, in each case based on the total weight of the fluid F2.

According to a particularly preferred embodiment of the process according to the invention, in process step ii) the untreated water-absorbing polymer structure is brought into contact with at most 20 wt. %, particularly preferably at most 15 wt. %, still more preferably at most 10 wt. % and more preferably at most 5 wt. %, but preferably with at least 1 wt. %, particularly preferably with at least 2 wt. %, more preferably with at least 3 wt. % and most preferably at least 4 wt. % of a solvent, preferably water, the above-mentioned wt. % data being based on the weight of the untreated water-absorbing polymer structure.

It is furthermore preferable according to the invention for the untreated water-absorbing polymer structure to be brought into contact in process step ii) with 0.1 to 20 wt. %, particularly preferably 0.5 to 15 wt. %, more preferably 1 to 10 wt. % and most preferably 2.5 to 7.5 wt. % of the acidic component, in each case based on the weight of the untreated water-absorbing polymer structure provided in process step i).

It may furthermore be advantageous for the untreated water-absorbing polymer structure to be brought into contact with the acidic component in process step ii) at a temperature in a range of from 30 to 210° C., particularly preferably from 40 to 150° C. and most preferably in a range of from 50 to 100° C. It is also conceivable for the untreated water-absorbing polymer structure to be brought into contact with the acidic component at a lower temperature, for example at room temperature, and for the mixture obtained in this way only then to be heated to the above-mentioned temperatures.

According to a particularly preferred embodiment of the process according to the invention, in process step ii) the untreated water-absorbing polymer structure is also additionally brought into contact with, in addition to the acidic, preferably organic component, an inorganic component which differs from the acidic component. This inorganic component is preferably an inorganic component which contains silicon and oxygen and, according to a particular embodiment of the process according to the invention, is present in the form of a powder.

Preferred inorganic components containing silicon and oxygen include compounds which are obtainable by polycondensation of mono-orthosilicic acid, and silicates. Particularly preferred polysilicic acids are silica sols such as are described in DE 102 49 821, which is introduced herewith as reference and the disclosure of which with respect to the silica sols is part of the disclosure of the present invention. Among the silicates, three-dimensional silicates, such as zeolites or silicates which have been obtained by drying aqueous silica solutions or silica sols, for example the commercially obtainable pyrogenic silicas known by the name Aerosil®, which preferably have a particle size in the range of from 5 to 50 nm, particularly preferably in the range of from 8 to 20 mil, are preferred in particular. Precipitated silicas, in particular the precipitated silicas known by the name Sipernat®, are also possible. Preferred silicates are furthermore all the natural or synthetic silicates which are disclosed as silicates in “Holleman and Wiberg, Lehrbuch der Anorganischen Chemie [Textbook of Inorganic Chemistry], Walter de Gruyter-Verlag, 91st-100th edition, 1985” on pages 750 to 783. The above-mentioned section of this textbook is introduced herewith as reference and is part of the disclosure of the present invention.

Particularly preferred zeolites are natural zeolites from the natrolite group, the harmotome group, the mordenite group, the chabasite group, the faujasite group (sodalite group) or the analcite group. Examples of natural zeolites are analcime, leucite, pollucites, wairakites, bellbergites, bikitaites, boggsites, brewsterites, chabasite, willhendersonites, cowlesites, dachiardites, edingtonite, epistilbite, erionite, fauj asite, ferrierites, amicites, garronites, gismondines, gobbinsites, grnelinite, gonnardites, goosecreekite, harmotome, phillipsite, wellsites, clinoptilolite, heulandite, laumontite, levynes, mazzites, merlinoites, montesonmlaites, mordenite, mesolite, natrolite, scolecite, offretites, paranatrolites, paulingites, perlialites, barrerites, stilbite, stellerite, thomsonite, tschernichites or yugawaralites. Preferred synthetic zeolites are zeolite A, zeolite X, zeolite Y, zeolite P or the product ABS CENTS.

Among the inorganic components containing silicon and oxygen, however, pyrogenic silica, such as is obtainable, for example, under the trade name Aerosil®, silica sol, such as is obtainable, for example, under the trade name Levasil®, or precipitated silica, such as is obtainable, for example, under the trade name Sipernat®, is preferred.

In connection with this particular embodiment of the process according to the invention, it is furthermore preferable for the untreated water-absorbing polymer structure to be brought into contact in process step ii) with 0.001 to 5 wt. %, particularly preferably 0.01 to 2.5 wt. % and most preferably 0.1 to 1 wt. % of the inorganic component, in each case based on the weight of the untreated water-absorbing polymer structure provided in process step i).

If the untreated water-absorbing polymer structure is additionally brought into contact in process step ii) with the inorganic, preferably pulverulent component, in addition to the acidic, preferably organic component, various possibilities are conceivable for this bringing into contact:
according to a first variant, the untreated water-absorbing polymer structure optionally already post-crosslinked on the surface is first brought into contact with the acidic component, either in powder form or in the form of the fluid F2, preferably in the form of the fluid F2, and the mixture obtained in this way is then brought into contact with the inorganic, preferably pulverulent component;
according to a second and particularly preferred variant, the untreated water-absorbing polymer structure optionally already post-crosslinked on the surface is first brought into contact with the preferably pulverulent inorganic component and the mixture obtained in this way is then brought into contact with the acidic component, either in powder form or in the form of the fluid F2, preferably in the form of the fluid F2;
according to a third variant, the untreated water-absorbing polymer structure optionally already post-crosslinked on the surface is brought into contact simultaneously with the preferably pulverulent inorganic component and the acidic component, either in powder form or in the form of the fluid F2, preferably in the form of the fluid F2. In this case, the preferably pulverulent inorganic component and the acidic component preferably present in the form of the fluid F2 could be added separately to the untreated water-absorbing polymer structure. However, it is in principle also conceivable for the preferably pulverulent inorganic component first to be mixed with the acidic component preferably present in the form of the fluid F2 and for the mixture obtained in this way then to be brought into contact with the untreated water-absorbing polymer structure.

If, in particular, in the process according to the invention in process step ii) the water-absorbing polymer structure is brought into contact not only with the acidic but additionally also with the inorganic component, it is preferable for the acidic component to be employed in the form of the fluid F2 described above, in this case the water-absorbing polymer structures being brought into contact with at least 1 wt. %, particularly preferably with at least 2 wt. %, more preferably with at least 3 wt. % and most preferably at least 4 wt. % of the solvent in which the acidic component is dissolved or dispersed.

Process step ii) of the process according to the invention can also be followed by a process step
iii) further surface modification of the water-absorbing polymer structure obtained in process step ii).

If water-absorbing polymer structures which have not been post-crosslinked on the surface have been employed in process step i), this further surface modification can be surface post-crosslinking. Conceivable further surface modification is furthermore bringing the water-absorbing polymer structures obtained in process step ii) into contact with permeability-increasing agents, for example with salts. Preferred salts are phosphates or salts containing a polyvalent, preferably trivalent cation. Among these salts, particularly preferred salts are those containing chloride anions, iodide anions, bromide anions, nitrate anions, nitrite anions, sulfide anions, sulfite anions, sulfate anions, carbonate anions, bicarbonate anions, hydroxide anions or organic anions, such as acetate anions or oxalate anions. Particularly preferred salts containing a trivalent cation are aluminum chloride, polyaluminum chloride, aluminum sulfate, aluminum nitrate, aluminum potassium bis-sulfate, aluminum sodium bis-sulfate, aluminum lactate, aluminum oxalate, aluminum citrate, aluminum glyoxylate, aluminum succinate, aluminum itaconate, aluminum crotonate, aluminum butyrate, aluminum sorbate, aluminum malonate, aluminum benzoate, aluminum tartrate, aluminum pyruvate, aluminum valerate, aluminum formate, aluminum glutarate, aluminum propanoate and aluminum acetate, AICl3×6H2O, NaAl(SO4)2×12H2O, Al(NO3)3×9H2O, KAl(SO4)2×12H₂O or Al2(SO4)3×14-18H₂O and the corresponding anhydrous salts, Na2SO4 or hydrates thereof, MgSO4×10H₂O or anhydrous magnesium sulfate being most preferred.

This further surface modification can furthermore be bringing the water-absorbing polymer structure obtained in process step ii) into contact with a compound which reduces dust formation, such as, for example, a polyvinyl alcohol, or with a compound which is capable of binding odors, such as, for example, a cyclodextrin or a zeolite.

A further contribution towards achieving the above-mentioned objects is made by a water-absorbing polymer structure which is obtainable by the process described above. This water-absorbing polymer structure preferably comprises an inner region and an outer region surrounding the inner region, the outer region of the water-absorbing polymer structures having been brought into contact with the acidic component described above and optionally also with the inorganic, preferably pulverulent component described above. In this context, the water-absorbing polymer structure obtainable by the process according to the invention is characterized in particular in that it is inhomogeneous with respect to its degree of neutralization. In this context, “inhomogeneous with respect to its degree of neutralization” means that the outer region of the water-absorbing polymer structure has a lower degree of neutralization, preferably a degree of neutralization which is lower by at least 1%, particularly preferably by at least 2.5%, still more preferably by at least 5% and most preferably by at least 10%, than the inner region. For example, if the inner region has a degree of neutralization of 60 mol %, the degree of neutralization in the outer region is preferably at most 59 mol %, particularly preferably at most 57.5 mol %, still more preferably at most 55 mol % and most preferably at most 50 mol %.

In this context, it is furthermore preferable for the water-absorbing polymer structure obtainable by the process described above to have at least one, preferably all of the following properties:
(β1) a retention, determined in accordance with ERT 441.2-02 for the total particle fraction, of at least 27 g/g, particularly preferably at least 29 g/g, more preferably at least 31 g/g and most preferably at least 33 gig, a retention preferably of 50 g/g, particularly preferably 45 g/g and most preferably 40 g/g not being exceeded;
(β2) an SAP index of at least 140 cm3s/g, preferably of at least 160 cm3/g, more preferably of at least 180 cm3s/g and most preferably of at least 200 cm3/g, the SAP index being defined as follows

SAP index=(RET×SFC)/pH
and wherein
RET=the retention determined in accordance with ERT 441.2-02 for the total particle fraction,
SFC=the permeability determined in accordance with the test method described herein for the total particle fraction and
pH=the pH determined in accordance with ERT 400.2-02 for the total particle fraction;
(β3) an absorption, determined in accordance with ERT 442.2-02, under a pressure of 50 g/cm2 of at most 20 g/g, particularly preferably at most 19 g/g and most preferably at most 18 g/g, the absorption under a pressure of 50 g/cm2 preferably not falling below 5 g/g, particularly preferably 10 g/g and most preferably 15 g/g;
(β4) an ammonia-binding capacity, determined in accordance with the test method described herein, of at least 98 mg/g, particularly preferably of at least 99 mg/g and most preferably of at least 100 mg/g, an ammonia-binding capacity preferably of 130 mg/g, particularly preferably 120 mg/g and most preferably 110 mg/g not being exceeded;
(β5) a pH, determined in accordance with ERT 400.2-02 (in the case of particles, determined for the total particle size fraction), of less than 6.5, preferably less than 6.0, particularly preferably less than 5.5 and most preferably less than 5, the pH preferably not falling below 1.0, particularly preferably 2.0, more preferably 3.0 and most preferably 4.0.

Preferred water-absorbing polymer structures obtainable by the process according to the invention are those which are characterized by the following properties or combination of properties: ((31), ((32), (01)(02), ((31)(,62)033), ((31)02)(04), ((31)((32)035) and (01)(02)(03)(04)(05), the combination (01)(02)((35), in particular with a retention of at least 27 g/g and a pH of less than 5.5, being most preferred.

A contribution towards achieving the above-mentioned object is also made by a water-absorbing polymer structure comprising an inner region and an outer region surrounding the inner region, the outer region of the water-absorbing polymer structure having been brought into contact with the acidic component described above and optionally also with the inorganic, preferably pulverulent component described above, and the water-absorbing polymer structure having at least one, preferably all of the following properties:
(β1) a retention, determined in accordance with ERT 441.2-02, of at least 27 g/g, particularly preferably at least 29 g/g, more preferably at least 31 g/g and most preferably at least 33 g/g, a retention preferably of 50 g/g, particularly preferably 45 g/g and most preferably 40 g/g not being exceeded;
(β2) an SAP index (SAPI) of at least 140 cm3s/g, preferably of at least 160 cm3/g, more preferably of at least 180 cm3s/g and most preferably of at least 200 cm3/g, the SAP index being defined as follows

SAP index=(RET×SFC)/pH
and wherein
RET=the retention determined in accordance with ERT 441.2-02 for the total particle fraction,
SFC=the permeability determined in accordance with the test method described herein for the total particle fraction and
pH=the pH determined in accordance with ERT 400.2-02 for the total particle fraction;
(β3) an absorption, determined in accordance with ERT 442.2-02, under a pressure of 50 g/cm2 of at most 20 g/g, particularly preferably at most 19 g/g and most preferably at most 18 g/g, the absorption under a pressure of 50 g/cm2 preferably not falling below 5 g/g, particularly preferably 10 g/g and most preferably 15 g/g;
(β4) an ammonia-binding capacity, determined in accordance with the test method described herein, of at least 98 mg/g, particularly preferably of at least 99 mg/g and most preferably of at least 100 mg/g, an ammonia-binding capacity preferably of 130 mg/g, particularly preferably 120 mg/g and most preferably 110 mg/g not being exceeded;
(β5) a pH, determined in accordance with ERT 400.2-02 (in the case of particles, determined for the total particle size fraction), of less than 6.5, preferably less than 6.0, particularly preferably less than 5.5 and most preferably less than 5, the pH preferably not falling below 1.0, particularly preferably 2.0, more preferably 3.0 and most preferably 4.0.

Preferred water-absorbing polymer structures according to the invention are those which are characterized by the following properties or combination of properties: (β1), (β2), (β1)(β2), (β1)(β2)(β3), (β1)(β2)(β4), (δ1)(δ2)(β5) and (β1)(β2)(β3)(β4)(β5), the combination (β1)(β2)(β5), in particular with a retention of at least 27 g/g and a pH of less than 5.5, being most preferred.

A further contribution towards achieving the objects described above is made by a composite comprising the water-absorbing polymer structures according to the invention or the water-absorbing polymer structures obtainable by the process according to the invention (called—water-absorbing polymer structures according to the invention—in the following) and a substrate. In this context, it is preferable for the polymer structures according to the invention and the substrate to be firmly bonded to one another. Preferred substrates are foils of polymers, such as, for example, of polyethylene, polypropylene or polyamide, metals, nonwovens, fluff, tissues, woven fabric, natural or synthetic fibers, or other foams. It is furthermore preferable according to the invention for the composite to comprise at least one region which contains the water-absorbing polymer structure according to the invention in an amount in the range of from about 15 to 100 wt. %, preferably about 30 to 100 wt. %, particularly preferably from about 50 to 99.99 wt. %, furthermore preferably from about 60 to 99.99 wt. % and moreover preferably from about 70 to 99 wt. %, in each case based on the total weight of the region in question in the composite, this region preferably have a size of at least 0.01 cm3, preferably at least 0.1 cm3 and most preferably at least 0.5 cm3.

In a particularly preferred embodiment of the composite according to the invention, it is a sheet-like composite such as is described in WO 02/056812 A1 as “absorbent material”. The disclosure content of WO 02/056812 A1, in particular with respect to the precise structure of the composite, the weight of its constituents per unit area and its thickness, is introduced herewith as reference and represents a part of the disclosure of the present invention.

A further contribution towards achieving the above-mentioned objects is provided by a process for the production of a composite, wherein the water-absorbing polymer structures according to the invention and a substrate and optionally an additive are brought into contact with one another. Substrates which are employed are preferably those substrates which have already been mentioned above in connection with the composite according to the invention.

A contribution towards achieving the above-mentioned objects is also made by a composite obtainable by the process described above, this composite preferably have the same properties as the composite according to the invention described above.

A further contribution towards achieving the above-mentioned objects is made by chemical products comprising the polymer structures according to the invention or a composite according to the invention. Preferred chemical products are, in particular, foams, shaped articles, fibers, foils, films, cables, sealing materials, liquid-absorbing hygiene articles, in particular nappies and sanitary towels, carriers for plant or fungal growth-regulating agents or plant protection active compounds, additives for building materials, packaging materials or soil additives.

The use of the polymer structures according to the invention or of the composite according to the invention in chemical products, preferably in the above-mentioned chemical products, in particular in hygiene articles, such as nappies or sanitary towels, and the use of the superabsorber particles as carriers for plant or fungal growth-regulating agents or plant protection active compounds make a contribution towards achieving the above-mentioned objects. In the case of the use as carriers for plant or fungal growth-regulating agents or plant protection active compounds, it is preferable for the plant or fungal growth-regulating agents or plant protection active compounds to be able to be released over a period of time controlled by the carrier.

The invention is now explained in more details with the aid of test methods and non-limiting examples.

### Test Methods

### Determination of the SFC Value

The determination of the SFC value was carried out in accordance with the test method described in WO 95/26209 A1.

### Determination of the Ammonia-Binding Capacity

85 ml of a 0.9 wt. % strength sodium chloride solution are initially introduced into a 200 ml conical flask which can be closed with a glass stopper and are stirred by means of a magnetic stirrer. 15 ml of a 0.1 molar NaOH solution which is free from carbonates are added to this solution by means of a burette. About 200 mg of the superabsorber to be analyzed are then weight out exactly and sprinkled into the solution in the conical flask. The conical flask is closed by means of a glass stopper and the composition obtained in this way is stirred at 500 revolutions per minute for 60 minutes.

The composition in subsequently filtered by means of a filter paper (Schwarzband from Schleicher & Schull) and 50 ml of the filtrate obtained in this way are subsequently titrated to the first end point by means of a 0.1 molar HCl solution on a Titroprocessor (Metrolun 670 from Metrohm GmbH & Co.). A corresponding solution (85 ml of 0.9 wt. % strength NaCl solution+15 ml of 0.1 molar NaOH solution) without superabsorber serves as the control value.

The ammonia-binding capacity is determined as follows:

$$w [ mg \text{/} g ] = \frac{{( {V}_{1} - {V}_{2} )}^{x} {c}^{x} {F}^{x} M}{m}$$

wherein
w is the ammonia-binding capacity,
V₁ is the consumption of HCl solution in ml for the control value,
V₂ is the consumption of HCl solution in ml for the solution with the superabsorber,
c is the concentration of the HCl solution (0.1 mol/1),
M is the molar mass of ammonia (17.03 g/mol),
F is the factor 2, calculated from the ratio 100 ml/50 ml and
m is the amount of superabsorber employed in g.

In each case 6 determinations are carried out and the ammonia-binding capacity is stated as the mean of these determinations.

### EXAMPLES

### 1. Preparation of Untreated Water-Absorbing Polymer Structures (Process Step i))

A monomer solution consisting of 2,400 g of acrylic acid, 1,332.2 g of NaOH (50% strength), 4,057.4 g of deionized water, 2.14 g of polyethylene glycol 300 diacrylate (with a content of active substance of 78.4 wt. %), 6.92 g of monoallylpolyethylene glycol 450 monoacrylic acid ester (with a content of active substance of 72.8 wt. %) and 65.36 g of polyethylene glycol 750 monomethacrylic acid ester methyl ether (with a content of active substance of 73.4 wt. %) was freed from dissolved oxygen by flushing with nitrogen and cooled to the starting temperature of 4° C. When the starting temperature was reached, the initiator solution (2.4 g of sodium peroxydisulphate in 77.6 g of H2O, 0.56 g of 30% strength hydrogen peroxide solution in 15.44 g of H2O and 0.12 g of ascorbic acid in 39.88 g of H2O) was added. When the final temperature of approx. 100° C. was reached, the gel formed was comminuted with a neat chopper and dried in a drying cabinet at 150° C. for 2 hours. The dried polymer was coarsely crushed, ground by means of a cutting mill SM 10 with a 2 mm sieve and sieved to a powder having a particle size of from 150 to 850 μm (=powder A).

### 2. Surface Post-Crosslinking (Still Process Step i))

Powder A was mixed with an aqueous solution consisting of ethylene carbonate (1 wt. %) based on powder A) and water (3 wt. %) based on powder A) in a laboratory mixer and the mixture was subsequently heated at 150° C. in an oven for a period of 30 minutes (=powder B).

### 3. Coating, According to the Invention, of the Powders

The amounts of dry Aerosil° 200 stated in the following table, the amounts of citric acid stated in the table and the amounts of water, as the solvent, stated in the table were added to powder B (all the wt. % data are based on the water-absorbing polymer structure). The citric acid was added to powder B in the form of a 50 wt. % strength citric acid solution by means of a syringe and a 0.9 mm cannula at 750 revolutions per minute. If both Aerosil° 200 and citric acid solution were added, the Aerosil° 200 was first stirred into powder B in the dry state and thereafter the mixture was homogenized on a roller bench for 30 minutes, and the citric acid solution, as described above, was subsequently added by means of a syringe.

| | Powder | Powder | Powder | Powder | Powder |
|---|---|---|---|---|---|
| | B | C | D | E | F |
| | (n.i.¹⁾) | (n.i.¹⁾) | (i.²⁾) | (i.²⁾) | (i.²⁾) |
| Aerosil ®200 | 0 | 0.5 | 0 | 0.5 | 1.0 |
| Citric acid | 0 | 0 | 5.0 | 5.0 | 5.0 |
| Water | 0 | 5.0 | 5.0 | 5.0 | 5.0 |
| pH. | 5.30 | 5.36 | 5.11 | 5.15 | 4.93 |
| Retention [g/g] | 29.8 | 30.8 | 29.0 | 29.0 | 29.7 |
| SAP index | 5.6 | 40.2 | 141.0 | 180.0 | 174.0 |
| Ammonia-binding | 95.1 | 83.3 | 100.3 | 100.5 | 99.7 |
| capacity [mg/g] | | | | | |

¹⁾n.i. = not according to the invention
²⁾i. = according to the invention

1-22. (canceled)

23. A process for the preparation of a particulate water-absorbing polymer structure, comprising the process steps of:
i) providing an untreated water-absorbing polymer structure wherein the structure is a particle wherein the untreated water-absorbing polymer structure has a degree of neutralization of at most 70 mol % and the particulate water-absorbing polymer structure has an average particle size in the range of 150 to 600 μm; and
ii) bringing the untreated particulate water-absorbing polymer structure into contact with from 2.5 wt % to 7.5 wt % based on the untreated particulate water-absorbing polymer structure of a an acidic component selected from anisic acid, benzoic acid, formic acid, valeric acid, citric acid, glyoxylic acid, glycollic acid, glycerol phosphoric acid, glutaric acid, chloroacetic acid, chloropropionic acid, cinnamic acid, succinic acid, acetic acid, tartaric acid, lactic acid, pyruvic acid, fumaric acid, propionic acid, 3-hydroxypropionic acid, malonic acid, butyric acid, isobutyric acid, imidinoacetic acid, malic acid, isothionic acid, methylmaleic acid, adipic acid, itaconic acid, crotonic acid, oxalic acid, salicylic acid, gluconic acid, gallic acid, sorbic acid or p-oxybenzoic acid, wherein the untreated particulate water-absorbing polymer structure is brought into contact with the acidic component at a temperature of from 50° C. to 100° C.;
and wherein the treated particulate water-absorbing polymer structure comprises an inner region and an outer region surrounding the inner region, wherein the inner region has a degree of neutralization of at least 60% or more and the outer region has a degree of neutralization of at least 10% less than the degree of neutralization of the inner core, and the particulate water-absorbing polymer structure has superabsorbent polymer (SAP) index of at least 140×10⁻⁷ cm³s/g, wherein the SAP index is defined as
SAP index=(RET×SFC)/pH
wherein
RET=the retention determined in accordance with ERT 441.2-02,
SFC=the permeability determined in accordance with the test method described herein and
pH=the pH determined in accordance with ERT 400.2-02.

24. The process according to claim 23, wherein the untreated particulate water-absorbing polymer structure provided in process step i) is post-crosslinked on the surface and the particulate water-absorbing polymer structure has a retention, determined in accordance with ERT 441.2-02, of from 25 to 40 g/g.

25. The process according to claim 23, further including the step of bringing the particulate water-absorbing polymer structure into contact with an inorganic component, which differs from the acidic component in process step ii), wherein the inorganic component is an inorganic compound comprising silicon and oxygen.

26. The process according to claim 23, comprising from about 0.001 to about 5 wt. % of the inorganic component, based on the weight of the untreated water-absorbing polymer structure provided in process step i).

27. The process according to claim 23, wherein the acidic component is an organic acid.

28. The process according to claim 27, wherein the organic acid is a monocarboxylic acid, a dicarboxylic acid or a tricarboxylic acid.

29. The process according to claim 27, wherein the organic acid is selected from citric acid or tartaric acid.

30. A particulate water-absorbing polymer structure of a process according to claim 23, wherein the particulate water-absorbing polymer structure has at least one of the following properties:
(β1) an absorption, determined in accordance with ERT 442.2-02, under a pressure of 50 g/cm² of at most 20 g/g;
(β2) an ammonia-binding capacity, determined in accordance with the test method described herein, of at least 98 mg/g; and
(β3) a pH, determined in accordance with ERT 400.2-02, of less than 6.5.

31. A particulate water-absorbing polymer structure comprising an inner region and an outer region surrounding the inner region, wherein the outer region of the particulate water-absorbing polymer structure has been brought into contact with from 2.5 wt % to 7.5 wt % based on the particulate water-absorbing polymer structure of a an acidic component and with an inorganic;
wherein the inner region has a degree of neutralization of 60 mol % or more and the outer region has a degree of neutralization of at least 10 mol % less than the degree of neutralization of the inner core; and wherein the water-absorbing polymer structure has the following properties:
(β1) a retention, determined in accordance with ERT 441.2-02, of at least 27 g/g;
(β2) an absorption, determined in accordance with ERT 442.2-02, under a pressure of 50 g/cm² of at most 20 g/g;
(β3) pH, determined in accordance with ERT 400.2-02, of less than 6.5 and
(β4) an ammonia-binding capacity, determined in accordance with the test method described herein, of at least 98 mg/g.

32. A composite comprising the particulate water-absorbing polymer structure according to claim 30 and a substrate.

33. A process for the production of a composite, wherein the particulate water-absorbing polymer structure according to claim 30 and a substrate and optionally an auxiliary substance are brought into contact with one another.

34. A composite of a process according to claim 33.

35. The process according to claim 23, wherein said inorganic compound is a powder.

36. The process according to claim 23, wherein the compound containing silicon and oxygen is silica and the organic acid is citric acid.
